(19) United States
(12) Patent Application Publication
(10) Pub. No.: US 2024/0337716 A1
(43) Pub. Date: Oct. 10, 2024

(54) METHODS, SYSTEMS, DEVICES, AND STORAGE MEDIA FOR DIFFUSION TENSOR IMAGING

(71) Applicant: WUHAN UNITED IMAGING LIFE SCIENCE INSTRUMENT CO., LTD., Wuhan (CN)

(72) Inventors: Meiling JI, Wuhan (CN); Renkuan ZHAI, Wuhan (CN)

(73) Assignee: WUHAN UNITED IMAGING LIFE SCIENCE INSTRUMENT CO., LTD., Wuhan (CN)

(21) Appl. No.: 18/744,671

(22) Filed: Jun. 16, 2024

Related U.S. Application Data
(63) Continuation of application No. PCT/CN2022/138050, filed on Dec. 9, 2022.

(30) Foreign Application Priority Data
Dec. 16, 2021 (CN) 202111561208.7

(51) Int. Cl. G01R 33/54; A61B 5/055

(52) U.S. Cl. CPC G01R 33/543; A61B 5/055

(57) ABSTRACT

Embodiments of the present disclosure provide a method, a system and a device for diffusion tensor imaging, and a storage medium. The method includes determining diffusion gradient directions; determining at least one scanning direction sequence based on the diffusion gradient directions, wherein each of the at least one scanning direction sequence includes at least one target scanning direction; and obtaining at least one set of scanning data by scanning a target object based on the at least one scanning direction sequence, wherein the at least one set of scanning data is used to determine a fiber tracking image of the target object.

### CROSS-REFERENCE TO RELATED APPLICATIONS

This application is a continuation of International Application No. PCT/CN2022/138050, filed on Dec. 9, 2022, which claims priority to Chinese Patent Application No. 202111561208.7, filed on Dec. 16, 2021, the entire contents of each of which are hereby incorporated by reference.

### TECHNICAL FIELD

The present disclosure relates to the field of medical imaging technology, and in particular, to methods, systems, devices, and storage media for diffusion tensor imaging.

### BACKGROUND

Nuclear magnetic resonance imaging (NMRI), also referred to as magnetic resonance imaging (MRI), is a widely used method for medical image acquisition. Diffusion tensor imaging (DTI) is an advanced application of the NMRI. Relatively good results can be obtained when imaging the brain using the DTI. The DTI can reveal neural network connections within the brain, which can not only show information such as “how a tumor affects nerve cell connections,” but also reflect subtle abnormal changes in the brain with stroke, multiple sclerosis, schizophrenia, dyslexia, etc. Therefore, the DTI has become one of the most important imaging applications for clinical and scientific research.

Currently, in order to obtain better fiber tracking effect, a method for diffusion tensor imaging scans a target object in separate directions of diffusion gradients in sequence, resulting in extremely high requirements on the stability of a magnetic resonance system. For example, if a field drift occurs, and a gradient temperature is too high, it may cause various errors and artifacts in an image. In extreme cases, the scan will fail, and a re-scan is needed. Prolonged high-resolution scanning leads to excessive data volume and scanning time, and post-processing is also required to correct data acquisition errors in order to ensure the accuracy of the final fiber tracking effect in the scanning, which causes the existing method for diffusion tensor imaging to be inefficient, and difficult to ensure the quality of image reconstruction results.

Accordingly, it is desired to provide a method, a system, a device, and a storage medium for diffusion tensor imaging, so as to improve the efficiency of the diffusion tensor imaging.

### SUMMARY

Some embodiments of the present disclosure provide a method for diffusion tensor imaging. The method may include determining diffusion gradient directions. The method may include determining at least one scanning direction sequence based on the diffusion gradient directions. Each of the at least one scanning direction sequence may include at least one target scanning direction. The method may further include obtaining at least one set of scanning data by scanning a target object based on the at least one scanning direction sequence. The at least one set of scanning data may be used to determine a fiber tracking image of the target object.

In some embodiments, the determining at least one scanning direction sequence based on the diffusion gradient directions may include obtaining a user instruction, wherein the user instruction includes indicative information of the at least one target scanning direction; and determining the at least one scanning direction sequence based on the user instruction.

In some embodiments, the diffusion gradient directions may be determined based on a total count of the diffusion gradient directions, and the method may further include generating a distribution map of the diffusion gradient directions in response to obtaining the total count of the diffusion gradient directions; and annotating, based on the user instruction, the at least one target scanning direction on the distribution map.

In some embodiments, the user instruction may include the at least one target scanning direction which is selected by a user on the distribution map.

In some embodiments, the determining at least one scanning direction sequence based on the diffusion gradient directions may include obtaining a count of the at least one target scanning direction input by a user; determining at least one set of candidate scanning directions from the diffusion gradient directions based on the count of the at least one target scanning direction; recommending the at least one set of candidate scanning directions to the user; and determining the at least one scanning direction sequence in response to a selection of the user.

In some embodiments, the method for diffusion tensor imaging may further include determining a current scanning direction; obtaining current scanning data corresponding to the current scanning direction; obtaining correction parameters based on the current scanning data; and correcting, based on the correction parameters, initial parameters for a next scanning.

In some embodiments, the method for diffusion tensor imaging may further include obtaining at least two diffusion factors, wherein the at least two diffusion factors include at least one zero-valued diffusion factor and at least one non-zero diffusion factor, or at least two non-zero diffusion factors. The scanning a target object may further include scanning the target object based on the at least two diffusion factors.

In some embodiments, the at least one non-zero diffusion factor may include a first diffusion factor and a second diffusion factor. The scanning the target object based on the at least two diffusion factors may include obtaining the at least one set of scanning data by scanning the target object based on the at least one scanning direction sequence and at least one of the first diffusion factor and the second diffusion factor.

In some embodiments, the method for diffusion tensor imaging may further include obtaining a plurality of reconstructed images by performing data reconstruction based on each of the at least one set of scanning data; and determining the fiber tracking image of the target object based on reconstructed images of at least six directions among the plurality of reconstructed images.

In some embodiments, the reconstructed images of the at least six directions may be obtained by reconstructing the at least one set of scanning data including a plurality of diffusion factor values in at least six different target scanning directions.

In some embodiments, the plurality of reconstructed images may be stored based on the at least one scanning direction sequence and at least one diffusion factor.

In some embodiments, the determining the fiber tracking image of the target object based on reconstructed images of at least six directions among the plurality of reconstructed images may include obtaining at least one pre-selected reconstructed images selected by a user from the plurality of reconstructed images; determining whether the at least one pre-selected reconstructed image satisfies a preset condition; in response to determining that the at least one pre-selected reconstructed image satisfies the preset condition, determining the fiber tracking image of the target object based on the at least one pre-selected reconstructed image; or in response to determining that the at least one pre-selected reconstructed image does not satisfy the preset condition, outputting a prompt message.

In some embodiments, the preset condition may include a count of diffusion gradient directions corresponding to the at least one pre-selected reconstructed image being greater than or equal to a preset value, and the diffusion gradient directions corresponding to the at least one pre-selected reconstructed image being uniformly distributed on a sphere surface.

Some embodiments of the present disclosure provide a system for diffusion tensor imaging. The system may include a diffusion gradient direction determination module, a scanning direction sequence determination module, and a scanning module. The diffusion gradient direction determination module may be configured to determine diffusion gradient directions. The scanning direction sequence determination module may be configured to determine at least one scanning direction sequence based on the diffusion gradient directions. Each of the at least one scanning direction sequence may include at least one target scanning direction. The scanning module may be configured to obtain at least one set of scanning data by scanning a target object based on the at least one scanning direction sequence. The at least one set of scanning data may be used to determine a fiber tracking image of the target object.

Some embodiments of the present disclosure provide a magnetic resonance imaging (MRI) system, including a processor and a magnetic resonance scanning device. The processor may be configured to present a user interaction interface at a display device, and the magnetic resonance scanning device may be configured to scan a target object based on an instruction from the processor. The user interaction interface may include a scanning setup module, and the scanning setup module may at least include a diffusion gradient direction setup unit, a diffusion factor setup unit, and a scanning mode setup unit. The diffusion gradient direction setup unit may be configured to set a total count of diffusion gradient directions. The diffusion factor setup unit may be configured to set diffusion factor parameters, the diffusion factor parameters may at least include a count of diffusion factors and diffusion factor values. The scanning mode setup unit may be configured to set scanning mode information, the scanning mode information may at least include whether to scan in separate directions and a current scanning direction.

In some embodiments, the scanning setup module may further include a presentation unit, and the presentation unit may be configured to display a distribution map of diffusion gradient directions and at least one target scanning direction selected by a user.

Some embodiments of the present disclosure provide a device for diffusion tensor imaging, including a storage and a processor. The storage may store a computer program, and the processor may execute the computer program to implement the method for diffusion tensor imaging.

Some embodiments of the present disclosure provide a device for diffusion tensor imaging, comprising a processor. The processor may be configured to implement the method for diffusion tensor imaging.

Some embodiments of the present disclosure provide a non-transitory computer-readable storage medium storing computer instructions. When a computer reads the computer instructions in the storage medium, the computer may implement the method for diffusion tensor imaging.

In some embodiments of the present disclosure, diffusion tensor scanning can be performed in separate directions and times. A plurality of required diffusion gradient directions can be divided into a plurality of sequences for scanning according to needs, thereby reducing data volume obtained in each scan, shortening a reconstruction time, reducing image errors and artifacts, improving scanning stability, reducing post-processing requirements, and reducing reconstruction pressure. At the same time, supplementary scanning can be performed in required direction(s) according to the fiber tracking effect instead of re-scanning in all directions, which ensures the accuracy of the fiber tracking effect and improves the efficiency of image acquisition and reconstruction. The scanning can be performed based on required scanning direction(s) selected according to user requirements, which satisfies diversified requirements of different users, and avoids loss of time, experimental materials, and other materials caused by the re-scanning when the scanning fails over a long period of time, thereby saving costs and improving economic efficiency.

### BRIEF DESCRIPTION OF THE DRAWINGS

The present disclosure will be further illustrated by way of exemplary embodiments, which will be described in detail with reference to according to the drawings. These embodiments are not limiting, and in these embodiments the same numbering indicates the same structure, wherein:

FIG. 1 is a schematic diagram illustrating an application scenario of a system for diffusion tensor imaging according to some embodiments of the present disclosure;

FIG. 2 is a schematic diagram illustrating a system for diffusion tensor imaging according to some embodiments of the present disclosure;

FIG. 3 is a flowchart illustrating an exemplary process for diffusion tensor imaging according to some embodiments of the present disclosure;

FIG. 4 is a flowchart illustrating an exemplary process for diffusion tensor imaging according to some other embodiments of the present disclosure;

FIG. 5 is a flowchart illustrating an exemplary process for diffusion tensor imaging according to some other embodiments of the present disclosure;

FIG. 6 is a flowchart illustrating an exemplary process for diffusion tensor imaging according to some other embodiments of the present disclosure;

FIG. 7 is a flowchart illustrating an exemplary process for diffusion tensor imaging according to some other embodiments of the present disclosure;

FIG. 8 is a schematic diagram illustrating an exemplary process for diffusion tensor imaging according to some embodiments of the present disclosure;

FIG. 9 is a schematic diagram illustrating a scanning setup interface according to some embodiments of the present disclosure;

FIG. 10 is a schematic diagram illustrating a distribution of diffusion gradient directions according to some embodiments of the present disclosure;

FIG. 11 is a schematic diagram illustrating a structure of a device for diffusion tensor imaging according to some embodiments of the present disclosure; and

FIG. 12 is a schematic diagram illustrating a magnetic resonance imaging (MRI) system according to some embodiments of the present disclosure.

### DETAILED DESCRIPTION

In order to more clearly illustrate the technical solutions of the embodiments of the present disclosure, the accompanying drawings to be used in the description of the embodiments will be briefly described below. Obviously, the accompanying drawings in the following description are only some examples or embodiments of the present disclosure, and that the present disclosure may be applied to other similar scenarios in accordance with these drawings without creative labor for those of ordinary skill in the art. Unless obviously obtained from the context or the context illustrates otherwise, the same numeral in the drawings refers to the same structure or operation.

It should be understood that “system,” “device,” “unit,” and/or “module” as used herein is a way to distinguish between different components, elements, parts, sections, or assemblies at different levels. However, these words may be replaced by other expressions if other words accomplish the same purpose.

As indicated in the present disclosure and in the claims, unless the context clearly suggests an exception, the words “one,” “a,” “a kind of,” and/or “the” do not refer specifically to the singular but may also include the plural. In general, the terms “including” and “comprising” suggest only the inclusion of clearly identified steps and elements, which do not constitute an exclusive list, and the method or device may also include other steps or elements.

The present disclosure uses flowcharts to illustrate the operations performed by the system according to some embodiments of the present disclosure. It should be understood that the operations described herein are not necessarily executed in a specific order. Instead, they may be executed in reverse order or simultaneously. Additionally, other operations may be added to these processes or certain steps may be removed.

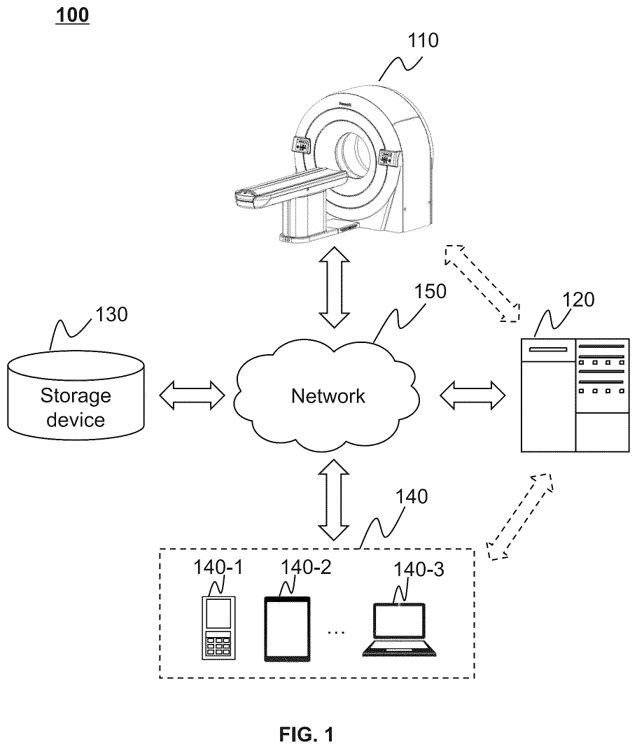

FIG. 1 is a schematic diagram illustrating an application scenario of a system 100 for diffusion tensor imaging according to some embodiments of the present disclosure.

As shown in FIG. 1, in some embodiments, the system 100 may include a medical imaging device 110, a processing device 120, a storage device 130, a terminal 140, and a network 150.

The medical imaging device 110 refers to a device in medicine that uses different media to represent an internal structure of a target object (e.g., a human body, an animal, etc.) as an image. In some embodiments, the medical imaging device 110 may include any medical device based on magnetic resonance imaging (MRI) techniques, such as an MRI device, etc. The medical imaging device 110 provided above is merely provided for purposes of illustration, and does not limit the scope of the medical imaging device 110. The medical imaging device 110 may receive instructions, etc., sent by the processing device 120, and perform relevant operations according to the instructions. For example, the medical imaging device 110 may scan and image the target object (e.g., a human body, an animal, a phantom, etc.). In some embodiments, the medical imaging device 110 may send scanning data obtained by scanning the target object to the processing device 120. In some embodiments, the medical imaging device 110 may exchange data and/or information with other components (e.g., the processing device 120, the storage device 130, and the terminal 140) in the system 100 via the network 150. In some embodiments, the medical imaging device 110 may be directly connected to other components in the system 100. In some embodiments, one or more components (e.g., the processing device 120, and the storage device 130) of the system 100 may be included within the medical imaging device 110.

The processing device 120 may process data and/or information obtained from other devices or components of the system 100, and perform a method for diffusion tensor imaging as illustrated in some embodiments of the present disclosure based on the data, information, and/or processing results to accomplish one or more functions described in some embodiments of the present disclosure. For example, the processing device 120 may determine a fiber tracking image of the target object based on the scanning data of the target object of the medical imaging device 110. As another example, the processing device 120 may correct scanning parameters based on the scanning data of the target object of the medical imaging device 110. In some embodiments, the processing device 120 may obtain a user instruction from the terminal 140 to determine at least one scanning direction sequence based on the user instruction. In some embodiments, the processing device 120 may send the determined scanning direction sequence to the medical imaging device 110, so as to cause the medical imaging device 110 to scan the target object based on the scanning direction sequence. In some embodiments, the processing device 120 may obtain pre-stored data and/or information (e.g., a user-specified target scanning direction, etc.) from the storage device 130 for implementing the method for diffusion tensor imaging in some embodiments of the present disclosure. For example, the processing device 120 may determine the at least one scanning direction sequence based on the target scanning direction, etc. In some embodiments, the processing device 120 may be connected to the medical imaging device 110 via the network 150 or directly connected to the medical imaging device 110 for exchanging the data/or information. In some embodiments, the processing device 120 may be included within the medical imaging device 110.

In some embodiments, the processing device 120 may include one or more sub-processing devices (e.g., a single-core processing device or a multi-core processing device). Merely by way of example, the processing device 120 may include a central processing unit (CPU), an application-specific integrated circuit (ASIC), an application-specific instruction processor (ASIP), a graphics processor (GPU), a physical processor (PPU), a digital signal processor (DSP), a field-programmable gate array (FPGA), a programmable logic circuit (PLD), a controller, a microcontroller unit, a reduced instruction set computer (RISC), a microprocessor, or the like, or any combination thereof.

The storage device 130 may store data or information generated by other devices. In some embodiments, the storage device 130 may store data and/or information (e.g., the scanning data, etc.) obtained by the medical imaging device 110. In some embodiments, the storage device 130 may store data and/or information (e.g., the user instruction, the scanning direction sequence, etc.) before or after processed by the processing device 120. The storage device 130 may include one or more storage components, each of the storage components may be a stand-alone device or a portion of other devices (e.g., a portion of any of the medical imaging device 110, the processing device 120, and the terminal 140). The storage device 130 may be local, or implemented via the cloud.

The terminal 140 may control an operation of the medical imaging device 110. The physician may issue the user instruction to the medical imaging device 110 via the terminal 140, so as to cause the medical imaging device 110 to complete a specified operation, such as scanning and imaging a specified body part of a patient. In some embodiments, the terminal 140 may perform the method for diffusion tensor imaging in some embodiments of the present disclosure by sending the user instruction to the processing device 120. In some embodiments, the terminal 140 may receive the fiber tracking image and/or a reconstructed image of the target object from the processing device 120, which allows the physician to accurately determine a physical condition of the patient for performing an effective and targeted examination and/or treatment on the patient. In some embodiments, the at least one terminal 140 may be any device with an input and/or output function, such as a mobile device 140-1, a tablet computer 140-2, a laptop computer 140-3, a desktop computer, or the like, or any combination thereof. In some embodiments, the terminal 140 may be connected to the processing device 120 via the network 150 or directly connected to the processing device 120 for exchanging the data/or information. In some embodiments, the terminal 140 may be included within the medical imaging device 110.

The network 150 may connect various components of the system 100 and/or connect the system 100 to an external resource part. The network 150 may enable communications between the various components and communications between the various components and other components outside the registration system 100 to facilitate data and/or information exchange. In some embodiments, one or more components (e.g., the medical imaging device 110, the processing device 120, the storage device 130, and the terminal 140) of the system 100 may send the data and/or information to other components via the network 150. In some embodiments, the network 150 may be any one or more of a wired network or a wireless network.

It should be noted that the above description is merely provided for purposes of illustration, and is not intended to limit the scope of the present disclosure. For persons having ordinary skills in the art, various variations and modifications may be conducted under the teaching of the present disclosure. The features, structures, methods, and other features of the exemplary embodiments described in the present disclosure can be combined in various manners to obtain additional and/or alternative exemplary embodiments. For example, the processing device 120 may be run based on a cloud computing platform, such as a public cloud, a private cloud, a community cloud, a hybrid cloud, etc. However, such variations and modifications do not depart from the scope of the present disclosure.

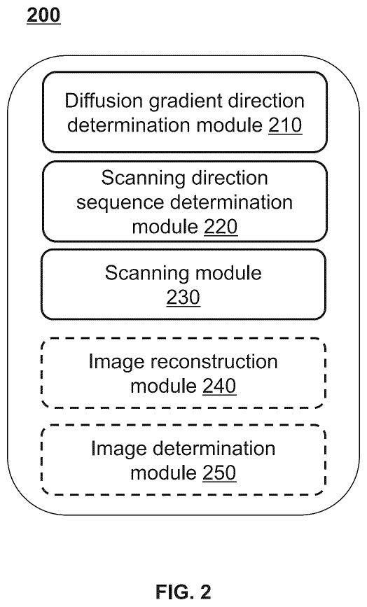

FIG. 2 is a schematic diagram illustrating a system 200 for diffusion tensor imaging according to some embodiments of the present disclosure.

As shown in FIG. 2, in some embodiments, the system 200 for diffusion tensor imaging may include a diffusion gradient direction determination module 210, a scanning direction sequence determination module 220, and a scanning module 230.

In some embodiments, the diffusion gradient direction determination module 210 may be configured to determine diffusion gradient directions.

In some embodiments, the diffusion gradient direction may be determined based on a total count of the diffusion gradient directions. The diffusion gradient direction determination module 210 may generate a distribution map (e.g., a gradient vector sphere map, etc.) of the diffusion gradient directions in response to obtaining the total count of the diffusion gradient directions.

In some embodiments, the scanning direction sequence determination module 220 may be configured to determine at least one scanning direction sequence based on the diffusion gradient directions. Each of the at least one scanning direction sequence may include at least one target scanning direction.

In some embodiments, the scanning direction sequence determination module 220 may obtain a user instruction, wherein the user instruction includes indicative information of the at least one target scanning direction; and determine the at least one scanning direction sequence based on the user instruction.

In some embodiments, the scanning direction sequence determination module 220 may annotate the at least one target scanning direction on the distribution map of the diffusion gradient directions based on the user instruction. The user instruction may include the at least one target scanning direction which is selected by a user on the distribution map.

In some embodiments, the scanning direction sequence determination module 220 may obtain a count of the at least one target scanning direction input by the user; determine at least one set of candidate scanning directions from the diffusion gradient directions based on the count of the at least one target scanning direction; recommend the at least one set of candidate scanning directions to the user; determine the at least one scanning direction sequence in response to a selection of the user.

In some embodiments, the system 200 may further include a diffusion factor acquisition module (not shown in FIG. 2). The diffusion factor acquisition module may be configured to obtain at least two diffusion factors. The at least two diffusion factors may include at least one zero-valued diffusion factor and at least one non-zero diffusion factor, or at least two non-zero diffusion factors.

In some embodiments, the scanning module 230 may be configured to obtain at least one set of scanning data by scanning a target object based on the at least one scanning direction sequence. The at least one set of scanning data may be used to determine a fiber tracking image of the target object.

In some embodiments, the scanning module 230 may also scan the target object based on the at least two diffusion factors.

In some embodiments, the at least one non-zero diffusion factor may include at least a first diffusion factor and a second diffusion factor. The scanning module 230 may obtain the at least one set of scanning data by scanning the target object based on the at least one scanning direction sequence and at least one of the first diffusion factor and the second diffusion factor.

In some embodiments, the system 200 may also include a correction module (not shown in FIG. 2). The correction module may be configured to determine a current scanning direction and obtain current scanning data corresponding to the current scanning direction; obtain correction parameters based on the current scanning data; and correct initial parameters for a next scanning based on the correction parameters.

In some embodiments, the system 200 may further include an image reconstruction module 240. The image reconstruction module 240 may be configured to obtain a plurality of reconstructed images by performing data reconstruction based on each of the at least one set of scanning data.

In some embodiments, the system 200 may further include an image determination module 250. The image determination module 250 may be configured to determine the fiber tracking image of the target object based on reconstructed images of at least six directions among the plurality of reconstructed images. The reconstructed images of the at least six directions may be obtained by reconstructing the at least one set of scanning data including a plurality of diffusion factor values in at least six different target scanning directions.

In some embodiments, the image determination module 250 may obtain at least one pre-selected reconstructed image selected by the user from the plurality of reconstructed images; determine whether the at least one pre-selected reconstructed image satisfies a preset condition; in response to determining that the at least one pre-selected reconstructed image satisfies the preset condition, determine the fiber tracking image of the target object based on the at least one pre-selected reconstructed image; or in response to determining that the at least one pre-selected reconstructed image does not satisfy the preset condition, output a prompt message.

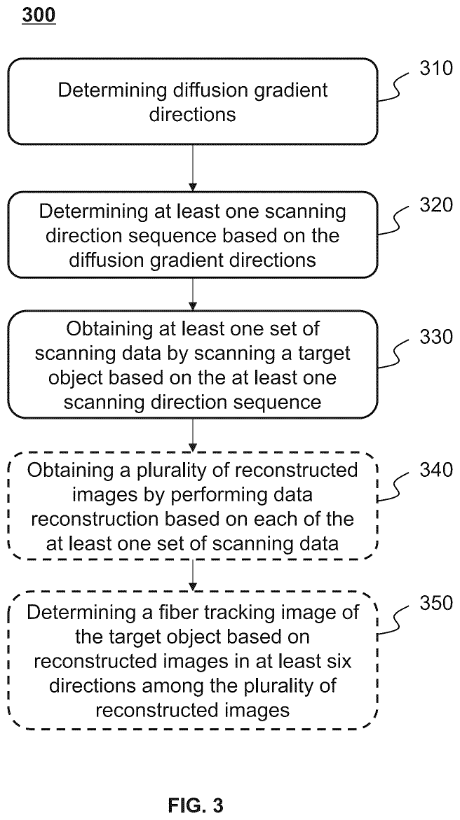

FIG. 3 is a flowchart illustrating an exemplary process 300 for diffusion tensor imaging according to some embodiments of the present disclosure.

As shown in FIG. 3, the process 300 may include following operations. In some embodiments, the process 300 may be performed by a processing device (e.g., the processing device 120).

In 310, diffusion gradient directions may be determined. In some embodiments, operation 310 may be performed by the diffusion gradient direction determination module 210.

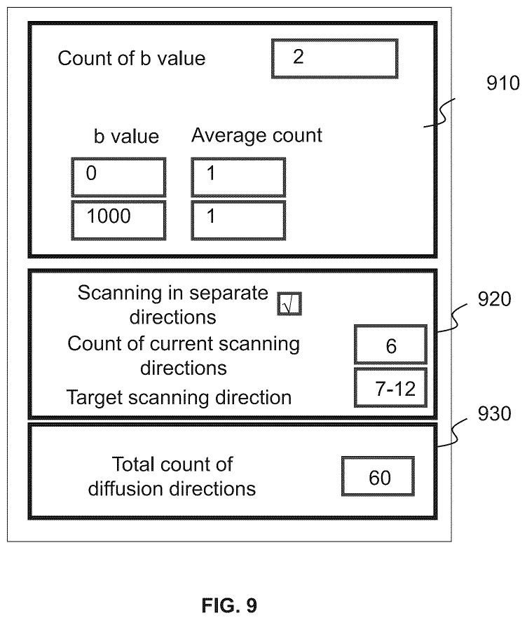

A diffusion gradient direction, also referred to as a diffusion direction, refers to a direction of a diffusion gradient when diffusion tensor scanning is performed on a target object (e.g., a human body, an animal, a phantom, etc.). In some embodiments, the diffusion gradient direction may be a scanning direction preset by a user. In some embodiments, the diffusion gradient directions may include a plurality of diffusion gradient directions, e.g., 6, 8, 12, etc. In some embodiments, in order to achieve normal fiber tracking during the diffusion tensor scanning, a count of the scanning directions may be greater than or equal to 6. Generally, the more scanning directions, the higher the precision, the more accurate the fiber tracking, and the better the scanning, but too many scanning directions may cause redundancy and increase unnecessary data volume and scanning duration. In some embodiments, the processing device may determine the count (i.e., a total count of the diffusion direction) of the diffusion directions (i.e., the diffusion gradient directions) by obtaining a user input. For example, as shown in FIG. 9, the user may select a customized diffusion direction option based on a preset scanning setup interface, and then input the total count of the diffusion directions in a preset direction count input region. The processing device may obtain the total count of the diffusion directions. As another example, the user may select a default gradient setup option based on the preset scanning setup interface, and the processing device may automatically select a preset default gradient direction table. As yet another example, the user may input a gradient direction table in a preset gradient direction table input region, and the processing device may update the total count of the diffusion directions needed to be scanned in real time after obtaining the gradient direction table.

In some embodiments, the processing device may determine the diffusion gradient directions based on the total count of the diffusion directions. In some embodiments, after determining the diffusion gradient directions, the processing device may generate a distribution map of the diffusion gradient directions. The distribution map may represent a spatial distribution of all the diffusion gradient directions.

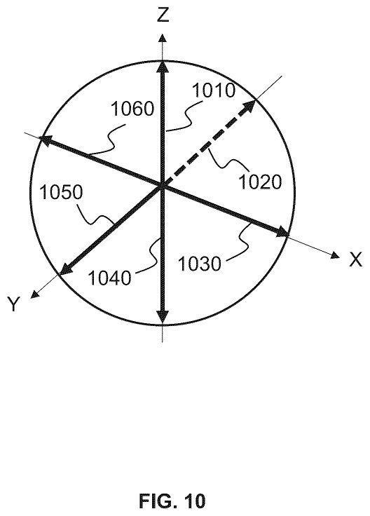

In some embodiments, the distribution map of the diffusion gradient directions may include a vector sphere uniformly distributed in a three-dimensional space, and the distribution map of the diffusion gradient directions may be referred to as a gradient vector sphere map. Vector arrows on a gradient vector sphere may be used to indicate the diffusion gradient directions. For example, in a gradient vector sphere map as shown in FIG. 10, directions shown by vector arrows 1010-1060 are diffusion gradient directions. The user may select a direction to be scanned (i.e., a target scanning direction) from the diffusion gradient directions, and the selected diffusion gradient direction may be displayed differently. For example, the vector arrow 1020 displayed by a dashed line in FIG. 10 is the target scanning direction selected by the user.

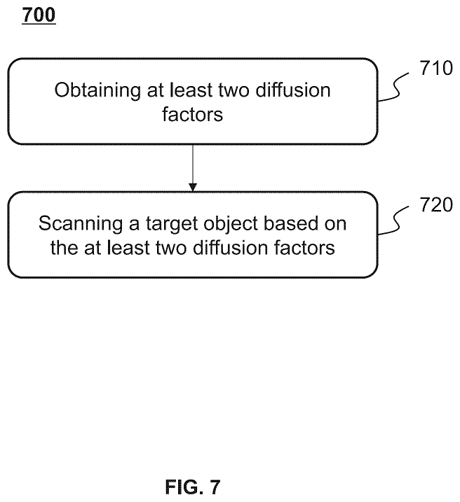

In some embodiments, the processing device may also determine at least one diffusion factor. The at least one diffusion factor may be denoted by b, a value of the at least one diffusion factor may affect the diffusion weighting. The larger the at least one diffusion factor, the heavier the diffusion weighting, the better contrast between different tissues, and the lower signal-to-noise ratio of the signal. In some embodiments, the count of the at least one diffusion factor may relate to an application scenario, and a plurality of images with different diffusion factor values may be fitted in some application scenarios. In some embodiments, the processing device may determine the count of the at least one diffusion factor, the value of each of the at least one diffusion factor (also referred to as at least one diffusion factor value), etc., by obtaining a user instruction, etc. In some embodiments, the user may set the count of the at least one diffusion factor greater than or equal to 2. In some embodiments, after setting the count of the at least one diffusion factor, the user may set a specific value for each diffusion factor. In some embodiments, the at least one diffusion factor may include at least two non-zero diffusion factors. That is, the at least one diffusion factor may include two or more diffusion factors, each of the two or more diffusion factors may be the non-zero diffusion factor. In some embodiments, the at least one diffusion factor may include a zero-valued diffusion factor and a non-zero diffusion factor. That is, the at least one diffusion factor may include two or more diffusion factors, and the at least one diffusion factor may include one or more zero-valued diffusion factors and at least one non-zero diffusion factor. In some embodiments, the user may set two diffusion factors. One of the two diffusion factors may take a value of 0, which indicates a diffusion situation without adding a diffusion gradient, and may be used as a baseline value for the calculation. The other one of the two diffusion factors may take a higher value, for example, 1000, 1500, etc. For example, as shown in FIG. 9, “b value” denotes at least one diffusion factor, a count of b value is two, one of the two b values is 0, and the other one of the two b values is 1000. Average counts corresponding to the two b values are set to 1. As the average count increases, the signal-to-noise ratio may increase. In some embodiments, after scanning the same target object multiple times, the processing device may perform a weighted average on data collected in the multiple scans with the same scanning direction and under the same diffusion factor value, and designate the weighted averaged data as an image in the scanning direction and under the diffusion factor value. More descriptions regarding the obtaining the at least one diffusion factor and the scanning based on the at least one diffusion factor may be found in other operations in the process 300 and relevant descriptions of FIG. 7, which are not repeated herein.

In 320, at least one scanning direction sequence may be determined based on the diffusion gradient directions. Each of the at least one scanning direction sequence may include at least one target scanning direction. In some embodiments, operation 320 may be performed by the scanning direction sequence determination module 220.

A scanning direction sequence refers to a diffusion gradient direction sequence including the target scanning direction(s), and a scanning device may scan the target object according to the target scanning direction(s) included in the scanning direction sequence. A target scanning direction refers to a scanning direction currently set by the user for the target object. In some embodiments, each scanning direction sequence may include scanning parameter information, such as the at least one target scanning direction, a count of scanning times, etc. In some embodiments, a portion of target scanning directions in different scanning direction sequences may be the same. For example, a scanning direction sequence A includes target scanning directions D1, D2, and D3, a scanning direction sequence B includes target scanning directions D2, D3, D4, and D5, and both the scanning direction sequences A and B include the target scanning directions D2 and D3.

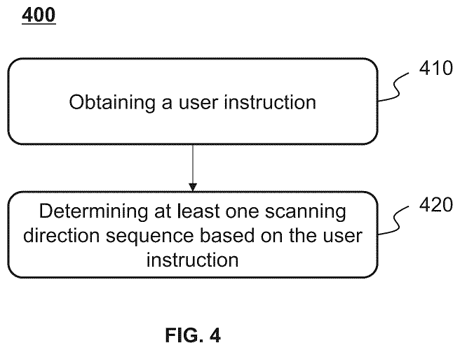

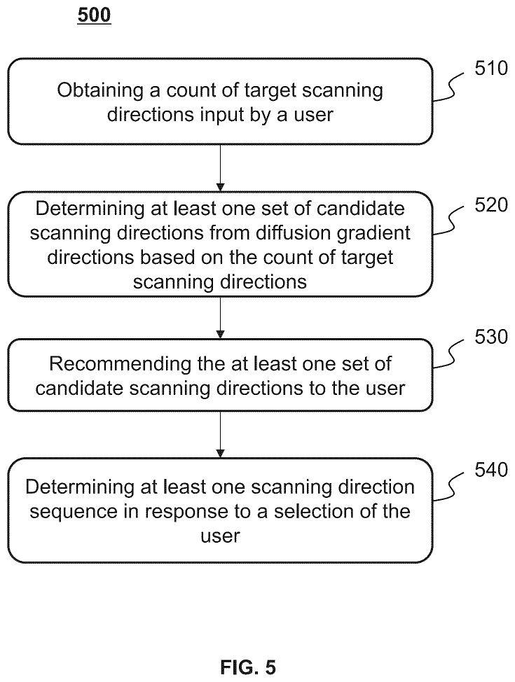

In some embodiments, after obtaining the diffusion gradient directions, the at least one scanning direction sequence may be determined based on the diffusion gradient directions through a plurality of manners. For example, the at least one scanning direction sequence may be determined by a user instruction, by selecting from candidate scanning directions, etc. More descriptions regarding the determination of the at least one scanning direction sequence based on the diffusion gradient directions may be found in FIGS. 4 and 5, and relevant descriptions thereof, which are not repeated herein.

In some embodiments, after obtaining the at least one diffusion direction, the user may further select a scanning direction grouping option based on the scanning setup interface to group the at least one diffusion direction in a preset selection region, as so to reconstruct images of different groups by scanning. Each group may include at least one target scanning direction, which forms a scanning direction sequence. In some embodiments, during the grouping process, a count of target scanning directions in each group may be the same (e.g., 8) or different (e.g., one group of 8 and another group of 6), which is not limited herein. For example, a total count of diffusion gradient directions may be 64, and every 8 consecutive diffusion directions may be determined as a group of target scanning directions. Alternatively, 8 diffusion gradient directions may be randomly selected as a group of target scanning directions. In some embodiments, the count of diffusion directions in each group of target scanning directions may not be limited. As another example, if the total count of obtained diffusion directions is 6, two diffusion directions among the diffusion directions may be selected as the target scanning directions based on the scanning setup interface, and the two target scanning directions may be further divided into two groups.

Alternatively, the two target scanning directions may be determined as one group. The above descriptions are merely provided as specific examples of the grouping, and do not limit the scope of the present disclosure. In some embodiments, different groups may include a portion of same target scanning directions. Therefore, different scanning direction sequences determined by these groups may include the portion of same target scanning directions. In some embodiments, different groups may include completely different target scanning directions. Therefore, different scanning direction sequences determined by the different groups may include completely different target scanning directions.

In some embodiments, the processing device may group the at least one target scanning direction in various manners, such as obtaining the user instruction, etc.

In some embodiments, the scanning setup interface may include a scanning grouping setup region, and the processing device may obtain a scanning direction grouping instruction via the scanning grouping setup region. The scanning direction grouping instruction includes a scanning direction grouping manner. For instance, the user may select the scanning grouping manner via the scanning grouping setup region of the scanning setup interface to trigger the scanning direction grouping instruction. The scanning grouping manner may include various manners, such as random grouping, manually assigning each group, etc. The above descriptions are merely provided by way of example, and do not limit the scope of the scanning direction grouping manner.

In some embodiments, after obtaining the at least one target scanning direction, the processing device may randomly group the at least one target scanning direction when obtaining the user instruction, which is triggered by the user by selecting an option of random grouping on the scanning setup interface. The random grouping may include average grouping and arbitrary grouping. For example, if the user selects a random average grouping manner, the processing device may randomly select different target scanning directions to divide into arbitrary groups, and the count of target scanning directions in each group may be the same. As another example, if the user selects a random arbitrary grouping manner, the processing device may randomly select different target scanning directions to divide into any groups, and the count of target scanning directions in each group may not be the same.

In some embodiments, after obtaining the at least one target scanning direction, the processing device may determine the target scanning direction(s) included in each group based on the scanning setup interface by obtaining the user instruction, etc. For example, the user may arbitrarily select different target scanning directions as one same group via the scanning grouping setup region, and the count of target scanning directions in each group may be the same or different. As shown in FIG. 9, a count of scanning directions included in a currently determined group (i.e., a scanning direction sequence) is six, and the current determined group includes target scanning directions numbered 7-12.

In some embodiments of the present disclosure, by obtaining the scanning direction grouping instruction via the scanning grouping setup region, the at least one target scanning direction can be grouped, so that the grouping can be performed in subsequent image reconstruction according to the at least one target scanning direction. Image reconstruction can be performed on scanning data obtained according to each group, which improves the efficiency of the image reconstruction.

In some embodiments, at the scanning setup interface, the processing device may recommend a grouping manner for gradient directions applicable for performing the fiber tracking based on a count of directions in each group set by the user. Therefore, the user may obtain a set of images that can be used individually for the fiber tracking after each set of scans. It should be noted that each set of images that can be used individually for the fiber tracking may also be used for the fiber tracking in combination with other sets of images. More descriptions regarding the recommending the gradient directions applicable for the fiber tracking based on the count of directions in each group set by the user may be found in FIG. 5 and relevant descriptions thereof, which are not repeated herein.

In some embodiments, after determining the groups of the at least one target scanning direction, the processing device may perform magnetic resonance scanning on the target object according to the groups of target scanning directions, respectively, and obtain scanning data corresponding to different groups.

In some embodiments, after grouping the at least one target scanning direction, the processing device may generate scanning direction sequences corresponding to a plurality of groups generated according to an increasing/decreasing order of serial number(s) of the at least one target scanning direction. In some embodiments, after obtaining the scanning direction sequences corresponding to the plurality of groups, the processing device may perform operation 330 and scan the target object in sequence according to the scanning direction sequences, thereby obtaining scanning data corresponding to the scanning direction sequences.

In 330, at least one set of scanning data may be obtained by scanning the target object based on the at least one scanning direction sequence. In some embodiments, operation 330 may be performed by the scanning module 230.

The scanning data refers to data obtained by scanning the target object, such as MRI data, etc. In some embodiments, after determining the at least one scanning direction sequence, for each of the at least one scanning direction sequence, the processing device may obtain information (e.g., the at least one target scanning direction, the count of scanning times, etc.), and obtain the at least one set of scanning data by scanning the target object according to the information. The grouping of the scanning data may be performed based on the at least one scanning direction sequence, the at least one target scanning direction, the at least one diffusion factor, etc. For example, each set of scanning data may correspond to data obtained by scanning based on a scanning direction sequence. As another example, each set of scanning data may correspond to data obtained by scanning based on a target scanning direction. Each set of scanning data corresponding to the data obtained by scanning based on the scanning direction sequence is described in the present disclosure as an example.

In some embodiments, after determining the groups of the at least one target scanning direction, scanning time between different groups may be sequential. That is, different groups may be scanned at different times, and a certain time interval may be between adjacent scans. In some embodiments, the processing device may correct system parameter(s) during group scanning. Alternatively, the processing device may re-scan in one or more directions along which the scanning effect is not good, only using re-scanning data to replace a corresponding portion in a set of scanning data corresponding to the group.

In some embodiments, the time interval between two sets of adjacent scans and/or two adjacent scans may be determined based on a state of the scanning device (e.g., a temperature of a gradient coil, etc.), user requirement(s) (e.g., a part to be imaged, a requirement for imaging quality, etc.), etc. In some embodiments, the processing device may automatically determine a recommended time interval, and present the recommended time interval to the user or prompt the user when the recommended time interval is reached.

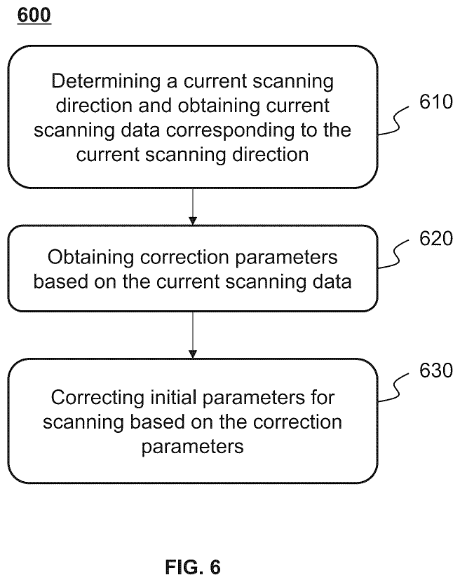

In some embodiments, in a process of scanning in different target scanning directions, the processing device may detect in real time whether the scanning generates scanning anomaly information, such as signal-to-noise information, eddy current correction information, etc. If the scanning anomaly information is generated, the processing device may adjust or recorrect setup parameter(s) (e.g., scanning-related system parameter(s), etc.) before scanning in a next target scanning direction, or before setting any target scanning directions for scanning, so as to avoid the generation of the scanning anomaly information. In such manner, it is not necessary to locate abnormal parameter(s) after completing the scanning in all directions, which effectively solves the problem of reduced scanning efficiency. More descriptions regarding the correcting the parameter(s) during the scanning based on the scanning data corresponding to the at least one target scanning direction may be found in FIG. 6 and relevant descriptions thereof, which are not repeated herein.

In some embodiments, after obtaining the grouped scanning data, the processing device may perform operations 340 and 350 to finally determine a fiber tracking image of the target object by reconstructing image(s) based on the scanning data.

In 340, a plurality of reconstructed images may be obtained by performing data reconstruction based on each of the at least one set of scanning data. In some embodiments, operation 340 may be performed by the image reconstruction module 240.

In some embodiments, for each set of the grouped scanning data, a preset image reconstruction algorithm (e.g., half-Fourier algorithm, compression perception algorithm, etc.) may be used to reconstruct the grouped scanning data, thereby obtaining a set of reconstructed images. The set of reconstructed images may include one or more reconstructed images, and each of the one or more reconstructed images may correspond to one scan. In some embodiments, at least six reconstructed images may be obtained after reconstructing each of the at least one set of scanning data.

In some embodiments, after grouping the at least one target scanning direction, when scanning in each group of scanning directions is completed, images corresponding to the group may be reconstructed according to the set of the scanning data using the preset image reconstruction algorithm. Therefore, images of different groups of target scanning directions may be obtained.

In order to ensure the proper fiber tracking effect, in some embodiments, the grouped scanning data used for data reconstruction may include scanning data corresponding to at least six different target scanning directions. For example, if the vector arrows 1010-1060 in FIG. 10 are determined as six target scanning directions of a scanning sequence, the scanning may be performed based on the directions identified by the vector arrows 1010-1060, the scanning data corresponding to the six target scanning directions may be obtained, and the data reconstruction may be performed based on the scanning data.

In some embodiments, the scanning data and/or the reconstructed images may be stored based on at least one of the at least one scanning direction sequence, the at least one diffusion factor, etc. The at least one scanning direction sequence may correspond to the at least one target scanning direction. For example, if a reconstructed image 1 corresponds to a scanning direction sequence S₁ and a diffusion factor B₁, the reconstructed image 1 may be classified under a category of “scanning direction sequence S₁ and diffusion factor B₁” when the reconstructed image 1 is stored. If a reconstructed image 2 corresponds to a scanning direction sequence S₂ and a diffusion factor B₂, the reconstructed image 2 may be classified under a category of “scanning direction sequence S₂ and diffusion factor B₂” when the reconstructed image 2 is stored. In some embodiments, scanning data and/or reconstructed images with different diffusion factors may be stored based on the at least one scanning direction sequence or the at least one target scanning direction. For example, reconstructed images corresponding to a same target scanning direction may be stored as a data set. As another example, reconstructed images corresponding to a same scanning direction sequence may be stored as a data set. By storing the scanning data and/or the reconstructed images in the above manner, when the image reconstruction is required, the same or similar category of data can be selected or required data can be filtered according to requirements.

In some embodiments, when a plurality of sets of scanning data are obtained, at least one reconstructed image may be obtained by performing the data reconstruction based on a portion of the plurality of sets of scanning data. For example, reconstructed images of at least six directions may be obtained based on a portion of three sets of scanning data.

In 350, the fiber tracking image of the target object may be determined based on reconstructed images in the at least six directions among the plurality of reconstructed images. In some embodiments, operation 350 may be performed by the image determination module 250.

The fiber tracking image refers to an image of fiber directions of an organ and/or a tissue (e.g., the brain, etc.) obtained using the process for diffusion tensor imaging. In some embodiments, the processing device may determine the fiber tracking image of the target object after processing the reconstructed images of the at least six directions among the plurality of reconstructed images. The reconstructed images of the at least six directions for determining the fiber tracking image of the target object may be obtained by reconstructing scanning data including a plurality of diffusion factor values (i.e., b values) in the at least six different target scanning directions. The scanning data may be derived from one set of scanning data or a plurality of sets of scanning data. In some embodiments, to perform the fiber tracking, the diffusion factor may include a zero value and at least one non-zero value. Scanning data corresponding to the at least one non-zero value may include data in the at least six different scanning directions. That is, in order to obtain the fiber tracking image, at least seven reconstructed images are required, including a reconstructed image corresponding to the zero-valued diffusion factor and reconstructed images of at least six directions corresponding to the at least one non-zero diffusion factor. For example, if a set D of scanning data includes non-zero diffusion factor(s) and six target scanning directions, reconstructed images including a combination of a reconstructed image set I_{D} generated based on the set D of scanning data with an image having the zero-valued diffusion factor may be obtained, and the reconstruction images may be used to determine the fiber tracking image of the target object. As another example, a set E of scanning data may include two target scanning directions, a set F of scanning data may include four target scanning directions that are different from the two target scanning directions in the set E, diffusion factors in the sets E and F may have a same non-zero value, and reconstructed image sets obtained based on the sets E and F of scanning data may be I_{E} and I_{F}, respectively. Neither the set I_{E} nor the set I_{F} can be used alone as the reconstructed images for determining the fiber tracking image of the target object, but reconstructed image(s) including a combination of the reconstructed image sets I_{E} and I_{F} and an image having a zero-valued diffusion factor may be used as the reconstructed images for determining the fiber tracking image of the target object. In some embodiments, in order to perform the fiber tracking, the at least one diffusion factor may include at least two non-zero values. The scanning data corresponding to each of the at least two non-zero values may include data in the at least six different scanning directions. In other words, in order to obtain the fiber tracking image, at least twelve reconstructed images may be obtained, and the at least twelve reconstructed images may include reconstructed images of the at least six directions corresponding to each non-zero diffusion factor. For example, if diffusion factors of sets G and H of scanning data are non-zero values, and diffusion factor values of the two sets are different, each of the sets G and H may include six target scanning directions, and reconstructed image sets obtained based on the sets G and E of scanning data may be I_{G} and I_{H}, respectively. A combination of the reconstructed image sets I_{G} and I_{H} may be used as reconstructed images for determining the fiber tracking image of the target object.

In order to select more suitable reconstructed image(s) to improve the effect of the fiber tracking image ultimately obtained, in some embodiments, the processing device may determine whether at least one pre-selected reconstructed image satisfies a preset condition, and determine the fiber tracking image of the target object based on the at least one pre-selected reconstructed image that satisfies the preset condition.

In some embodiments, the processing device may obtain the at least one pre-selected reconstructed image selected by the user from the plurality of reconstructed images, and determine whether the at least one pre-selected reconstructed image satisfies the preset condition. The at least one pre-selected reconstructed image may include a plurality of reconstructed images. In some embodiments, the plurality of pre-selected reconstructed images may include reconstructed images selected from different sets. In some embodiments, the preset condition may include a count of diffusion gradient directions corresponding to the at least one pre-selected reconstructed image being greater than or equal to a preset value, and diffusion gradient directions corresponding to the at least one pre-selected reconstructed image being uniformly distributed on a sphere surface. The preset value of the count of diffusion gradient directions may be set according to necessary conditions for the fiber tracking, user requirements, etc. For example, the preset value may be 6, 8, 12, etc. In some embodiments, to ensure the normal fiber tracking effect, the preset value of the count of diffusion gradient directions may be greater than or equal to six. That is, the at least one pre-selected reconstructed image may include at least six different directions. In some embodiments, for the plurality of reconstructed images with different diffusion factors, on the premise that the preset condition is satisfied, the reconstructed images may be designated as the pre-selected reconstructed images.

In some embodiments, the processing device may determine a spatial quadrant in which a diffusion gradient direction corresponding to each reconstructed image is located, and determine whether the pre-selected reconstructed images satisfy the preset condition based on a count of the spatial quadrant. For instance, when the count of the spatial quadrant is not less than 5, it may be determined that the at least one pre-selected reconstructed image satisfies the preset condition. When the count of the spatial quadrant is 3 or 4, it may be determined whether there exists a quadrant in the spatial quadrant that is not adjacent to at least another quadrant. If there exists the quadrant that is not adjacent to the at least another quadrant, the at least one pre-selected reconstructed image may satisfy the preset condition. Otherwise, it may be determined that the at least one pre-selected reconstructed image does not satisfy the preset condition. When the count of the spatial quadrant is less than or equal to 2, it may be determined that the at least one pre-selected reconstructed image does not satisfy the preset condition. In some embodiments, if the count of the spatial quadrant in which the diffusion gradient direction(s) corresponding to the at least one pre-selected reconstructed image satisfies the preset condition, the diffusion gradient direction(s) may be considered to be uniformly distributed. Therefore, the at least one pre-selected reconstructed image may be used to determine the fiber tracking image of the target object. In some embodiments, whether the diffusion gradient direction(s) are uniformly distributed on the sphere surface may be determined through a Fibonacci lattice point construction manner, a minimum potential energy manner, etc. In some embodiments, the processing device may determine the diffusion gradient direction(s) that have been set based on point coordinates of the sphere surface.

In some embodiments, when the at least one pre-selected reconstructed image satisfies the preset condition, the processing device may determine the fiber tracking image of the target object based on the at least one pre-selected reconstructed image. When the at least one pre-selected reconstructed image does not satisfy the preset condition, a prompt message (e.g., “The selected image does not satisfy the condition, please re-select,” etc.) may be output. In some embodiments, the user may obtain a combination of the diffusion gradient directions that satisfy the preset condition by re-selecting the reconstructed images.

In some embodiments, the processing device may select at least one set of images for the fiber tracking from the reconstructed images as the pre-selected reconstructed images, and obtain a fiber tracking result (i.e., the fiber tracking image) based on the pre-selected reconstructed images. The scanning data used to reconstruct the pre-selected reconstructed images may include a plurality of diffusion factor values in the at least six different target scanning directions.

In some embodiments, the processing device may obtain at least one synthesized fiber tracking image by processing the plurality of pre-selected reconstructed images. In some embodiments, the processing device may analyze overall scanning results based on different fiber tracking images, and designate an optimal fiber tracking image as the fiber tracking image of the target object.

In some embodiments, the processing device may directly designate a synthesized image obtained by processing the pre-selected reconstructed images as the fiber tracking image of the target object.

In some embodiments, the processing device may obtain a synthetic image obtained by processing reconstructed images obtained from a set of scanning data, and designate the synthetic image as the fiber tracking image of the target object. The set of scanning data may include a plurality of diffusion factor values and at least six target scanning directions. In some embodiments, a plurality of candidate fiber tracking images of the target object may be obtained based on a plurality of sets of scanning data, and then the plurality of candidate fiber tracking images may be synthesized to obtain a final candidate fiber tracking image of the target object.

In some embodiments, the processing device may also determine the fiber tracking image of the target object in other manners, e.g., through a machine learning model, etc.

In some embodiments of the present disclosure, the target scanning directions can be determined based on the total count of diffusion gradient directions set by the user, and the diffusion tensor scanning can be performed in separate directions and times. For the plurality of required diffusion directions, the target scanning directions can be divided into a plurality of sequences for scanning, thereby reducing the data volume in each image reconstruction, shortening the reconstruction time, reducing image errors and artifacts, improving scanning stability, reducing post-processing requirements, and reducing reconstruction pressure. By grouping the target scanning directions, the quality of the scanning data can be detected on time, and the scanning parameters can be adjusted on time when the scanning quality is poor, which can ensure the quality of the image. By selecting the required target scanning directions according to the user requirements, scanning in all the scanning directions can be avoided, and the re-scanning and reconstruction can only be performed in directions with problems, which can satisfy the requirements of different users, thereby reducing the pressure of scanning and reconstruction, and improving the efficiency. When the user selects the image used for the fiber tracking, whether the image satisfies the requirements can be prompted the user, which is more friendly to the user, thereby improving the user's satisfaction.

FIG. 4 is a flowchart illustrating an exemplary process 400 for diffusion tensor imaging according to some other embodiments of the present disclosure.

As shown in FIG. 4, the process 400 may include following operations. In some embodiments, the process 400 may be performed by a processing device (e.g., the processing device 120) or the scanning direction sequence determination module 220. In some embodiments, at least one scanning direction sequence may be determined based on diffusion gradient directions (i.e., operation 320) by performing the operations as shown in the process 400.

In 410, a user instruction may be obtained.

In some embodiments, the processing device may obtain the user instruction input by a user in a plurality of manners. The user instruction may include at least one indicative information of at least one target scanning direction, such as text information, interaction information of a graphical interface, etc. In some embodiments, the indicative information may include a selection manner (e.g., selecting default, scanning in separate directions, etc.) for the at least one target scanning direction. In some embodiments, the user instruction may include an instruction for setting scanning parameter(s), an instruction for operating a scanning device, etc. In some embodiments, the processing device may obtain the user instruction by obtaining an operation of the user on a scanning setup interface.

In 420, the at least one scanning direction sequence may be determined based on the user instruction.

In some embodiments, at the scanning setup interface, the processing device may obtain a total count of the diffusion gradient directions through the operation of the user, and generate a distribution map of the diffusion gradient directions. For example, at the scanning setup interface, the processing device may generate a gradient vector sphere map as shown in FIG. 10 based on the total count of the diffusion gradient directions set by the user, and present the gradient vector sphere map to the user (e.g., via a pop-up dialog box, etc.). More descriptions regarding the generation of the distribution map of the diffusion gradient directions may be found in relative descriptions in operation 310, which are not repeated herein.

In some embodiments, the user instruction may be selected as default. After determining the total count of diffusion gradient directions, the processing device may adopt default scanning direction(s) as the at least one target scanning direction, and determine the scanning direction sequence based on the default scanning direction(s). The default scanning direction(s) may include all or part of the diffusion gradient directions. In some embodiments, the processing device may scan sequentially according to an order (e.g., a clockwise order or a counterclockwise order on the gradient vector sphere map, etc.) of each diffusion gradient direction in the default scanning directions. For example, the user may select not to perform a separate direction scanning at the scanning setup interface.

In some embodiments, at the scanning setup interface, the processing device may obtain a separate direction scanning instruction by the operation of the user. The separate direction scanning instruction indicates that the user desires to divide the scanning into a plurality of directions or a plurality of sets of directions.

In some embodiments, the user instruction may be the separate direction scanning. At the scanning setup interface, after the user selects the separate direction scanning, the processing device may obtain the indicative information of the at least one target scanning direction from the user instruction, and determine the at least one scanning direction sequence based on the indicative information. For example, if the indicative information includes a target scanning direction, one or more scanning direction sequences including the target scanning direction may be determined. As another example, if the indicative information includes three target scanning directions, one or more scanning direction sequences may be determined. Each of the one or more scanning direction sequences may include at least one of the three target scanning directions, and the one or more scanning direction sequences may include all of the three target scanning directions.

In some embodiments, after generating the distribution map of the diffusion gradient directions based on the total count of the diffusion gradient directions, the processing device may annotate the at least one target scanning direction on the distribution map based on the user instruction.

In some embodiments, the user instruction may include that the user selects the at least one target scanning direction on the distribution map. In some embodiments, the user may select the at least one target scanning direction on the distribution map in a plurality of manners, for example, by tapping, directly inputting a serial number, etc. For example, as shown in FIG. 10, the user may select a vector arrow 1020 in the gradient vector sphere map (e.g., by annotating selected and unselected states with different colors) by clicking or by inputting the serial number, etc., and designate the direction that the vector arrow 1020 indicates as the target scanning direction.

In some embodiments, at the scanning setup interface, the user may directly input a current scanning direction and a count of scanning directions, and a vector (i.e., a vector arrow) in a corresponding direction in a corresponding gradient vector sphere map may be in the selected state. In some embodiments, the user may select a direction by directly tapping a gradient direction vector of the corresponding direction in the gradient vector sphere map, and the selected vector may be annotated.

In some embodiments of the present disclosure, by determining the scanning direction sequence based on the total count of the diffusion gradient directions input by the user, the operation of the user can be simplified, the scanning quality can be ensured, and the scanning efficiency can be improved. By graphically displaying the distribution map (e.g., the gradient vector sphere map) of the diffusion gradient directions, the user can intuitively obtain a distribution situation of the diffusion gradient direction(s) and determine the target scanning direction therefrom, which is intuitive and user-friendly in the operation.

FIG. 5 is a flowchart illustrating an exemplary process 500 for diffusion tensor imaging according to some other embodiments of the present disclosure.

As shown in FIG. 5, the process 500 may include following operations. In some embodiments, the process 500 may be performed by a processing device (e.g., the processing device 120) or the scanning direction sequence determination module 220. In some embodiments, at least one scanning direction sequence may be determined based on diffusion gradient directions (i.e., operation 320) by performing the operations as shown in the process 500.

In 510, a count of target scanning directions input by the user may be obtained.

In some embodiments, the processing device may obtain the count of target scanning directions input by the user using a user instruction, etc. In some embodiments, at a scanning setup interface, the processing device may obtain the count of target scanning directions input by the user by an operation of the user. For example, the user may directly input or select the count of target scanning directions. In some embodiments, the count of target scanning directions input by the user may be used to group the target scanning directions. The count of target scanning directions may be a count of target scanning directions included in each group.

In 520, at least one set of candidate scanning directions may be determined from the diffusion gradient directions based on the count of target scanning directions.

In some embodiments, the processing device may determine one or more sets of candidate scanning directions from the diffusion gradient directions based on the count of target scanning directions input by the user. Each of the one or more sets of candidate scanning directions may be combined with scanning data with a zero-valued diffusion factor to individually perform the fiber tracking on the target object. For example, if the count of target scanning directions input by the user is 6, the processing device may determine the one or more sets of candidate scanning directions based on the diffusion gradient directions determined in operation 310. Each of the one or more sets of candidate scanning directions may include 6 directions in the diffusion gradient directions. As shown in FIG. 9, a count of current scanning directions (i.e., the count of target scanning directions) input by the user is 6, and the processing device may determine at least one set of candidate scanning directions including 6 target scanning directions, which may include target scanning directions numbered 7-12.

In some embodiments, the processing device may determine an initial scanning direction based on the count of target scanning directions, and determine a first vector set based on the initial scanning direction and the count of target scanning directions. Each vector (i.e., a scanning direction vector) in the first vector set may be uniformly distributed in a three-dimensional space. For instance, the processing device may select a direction from the diffusion gradient directions as the initial scanning direction, and then determine positions of theoretical direction vectors that are theoretically uniformly distributed in the three-dimensional space based on the count of target scanning directions input by the user. The processing device may determine the initial scanning direction vector of the initial scanning direction and the theoretical direction vectors as the first vector set, then determine whether the initial scanning direction vector satisfies a preset condition based on the theoretical direction vectors. If the initial scanning direction vector satisfies the preset condition, the diffusion gradient direction (i.e., the initial scanning direction) corresponding to the initial scanning direction vector is determined as a candidate scanning direction. The preset condition may include a minimum angle between the theoretical direction vectors and the initial scanning direction vector being less than a preset threshold (e.g., 5°, etc.). In some embodiments, when a difference between minimum angles between adjacent direction vectors in a plurality of direction vectors in the three-dimensional space is less than a threshold (e.g., 1°, etc.) or equal to zero, the plurality of direction vectors may be considered to be uniformly distributed in the three-dimensional space. The adjacent direction vector of a direction vector refers to a direction vector that has a minimum angle with the direction vector.

In 530, at least one set of candidate scanning directions may be recommended to the user.

In some embodiments, the processing device may recommend the at least one set of candidate scanning directions to the user based on a preset rule. In some embodiments, the preset rules may include that the set may be directly used to perform the fiber tracking, etc. For example, the higher the requirement of the user on the fiber tracking effect, the smaller the minimum angle between the direction vector of the candidate scanning direction and the direction vector of the first vector set. As another example, scanning data for reconstructing an image set to perform the fiber tracking may include at least six scanning directions, and the at least six scanning directions may not be located in a same hemisphere of the gradient vector sphere. Stereoscopic angles between circles formed with corresponding points of any two adjacent directions on the gradient vector sphere as diameters are equal or approximately equal to a center of the gradient vector sphere. That is, the vectors in the candidate scanning directions should be distributed as uniformly as possible over the surface of the sphere. In some embodiments, each direction vector in a set of candidate scanning directions may have a first angle with an adjacent direction vector. When a difference of the first angle corresponding to each of the set of candidate scanning directions is less than a preset threshold (e.g., 3°, etc.), the direction vectors in the set of candidate scanning directions may be considered uniformly distributed.

In some embodiments, the candidate scanning directions may be grouped into one or more sets. For example, if a count of each set of directions set by the user is six, one or more sets of candidate scanning directions may be recommended to the user. Each of the one or more sets of candidate scanning directions may include at least six diffusion gradient directions. As shown in FIG. 9, the processing device may recommend the at least one set of candidate scanning directions including six target scanning directions to the user, and the target scanning directions numbered 7-12 may be included.

In 540, the at least one scanning direction sequence may be determined in response to a selection of the user.

In some embodiments, after recommending the candidate scanning directions to the user, the user may select from the candidate scanning directions. The at least one scanning direction sequence may be determined based on the one or more sets of candidate scanning directions selected by the user, and each of the one or more set of candidate scanning directions may correspond to one or more scanning direction sequences. For example, as shown in FIG. 9, the user may select a set of target scanning directions numbered 7-12 from recommended candidate scanning directions.

In some embodiments, the recommended candidate scanning directions may be displayed in the gradient vector sphere map according to sets, and the user may select one or more sets of candidate scanning directions from the gradient vector sphere map. Different sets may be distinguished from each other by colors, dashed lines, arrow shapes, etc.

In some embodiments of the present disclosure, by recommending the candidate scanning directions to the user based on the count of target scanning directions input by the user, the burden of the user can be reduced, and the difficulty of the operation can be lowered. At the same time, the quality of the scanning can be ensured, and the efficiency of the scanning can be improved.

FIG. 6 is a flowchart illustrating an exemplary process 600 for diffusion tensor imaging according to some other embodiments of the present disclosure.

As shown in FIG. 6, the process 600 may include following operations. In some embodiments, the process 600 may be performed by a processing device (e.g., the processing device 120). In some embodiments, when operation 330 is executed, initial parameter(s) during scanning may be corrected based on scanning data in one scanning by implementing the operations in the process 600.

In 610, a current scanning direction may be determined, and current scanning data corresponding to the current scanning direction may be obtained.

In some embodiments, the user may determine the current scanning direction from each target scanning direction based on a scanning setup interface. For instance, after obtaining the target scanning direction, according to actual user requirements, the user may input, based on the scanning setup interface, one set of target scanning directions that satisfies a count of target scanning directions in a current scanning direction setup region, and designate the set of target scanning directions as the current scanning direction.

In some embodiments, the processing device may obtain the current scanning data corresponding to the current scanning direction by scanning a target object (e.g., a human body, an animal, etc.) based on the current scanning direction. For instance, the processing device may generate, based on a pre-set protocol information, a scanning sequence of the current scanning direction according to the current scanning direction, and obtain the scanning data corresponding to the current scanning direction by scanning the target object according to the current scanning sequence.

In 620, correction parameters may be obtained based on the current scanning data.

In some embodiments, the processing device may determine whether scanning anomaly information (e.g., signal-to-noise ratio information, eddy current information, field drift information, etc.) is included in the current scanning data. If the scanning anomaly information is included, the processing device may generate the correction parameter for the scanning based on the scanning anomaly information.

In 630, initial parameters for scanning may be corrected based on the correction parameters.

In some embodiments, after generating the correction parameters for the scanning, the processing device may correct the scanning parameter used for a next scanning (i.e., the initial parameters during the scanning) based on the correction parameters before a next scanning (e.g., before scanning in a next target scanning direction, before setting another target scanning direction, etc.), and the corrected scanning parameter may be effective for the next scanning to avoid continuing to generate the scanning anomaly information.

In some embodiments of the present disclosure, a system state (e.g., eddy current, field drift, etc.) can be corrected by the scanning data of the diffusion tensor imaging in the at least one direction, and the obtained correction parameters can be used for the subsequent scanning, thereby improving the accuracy of scanning results. By correcting the scanning parameters in the interval between two scans, the problem of locating anomalous parameters only after finishing the scanning in all directions can be effectively solved, which improves the scanning efficiency.

FIG. 7 is a flowchart illustrating an exemplary process 700 for diffusion tensor imaging according to some other embodiments of the present disclosure.

As shown in FIG. 7, the process 700 may include following operations. In some embodiments, the process 700 may be performed by the processing device 120. In some embodiments, a target object may be scanned based on at least two diffusion factors by executing the operations as shown in the process 700 to obtain scanning data.

In 710, at least two diffusion factors may be obtained. The at least two diffusion factors may include at least one zero-valued diffusion factor and at least one non-zero diffusion factor, or at least two non-zero diffusion factors.

In some embodiments, the processing device may obtain the at least two diffusion factors by obtaining an operation or input of a user at a scanning setup interface. A target object may also be scanned based on the at least two diffusion factors. In some embodiments, the at least two diffusion factors may include at least one zero value and at least one non-zero value. The zero value indicates a diffusion situation without adding a diffusion gradient, and may be used as a baseline value for the calculation. In some embodiments, the at least two diffusion factors may exclude the zero value, i.e., all of the non-zero diffusion factors.

FIG. 9 is a schematic diagram illustrating a scanning setup interface according to some embodiments of the present disclosure. “B value” is a diffusion factor. In some embodiments, as shown in FIG. 9, a user may input a count of preset b values in a b value setup region based on the scanning setup interface. The count of preset b values is 2 in FIG. 9. After determining the count of preset b values, the user may set specific values of different preset b values, which are 0 and 1000 in FIG. 9, so as to obtain the preset b-values. That is, the diffusion factors are 0 and 1000.

In some embodiments, the non-zero diffusion factor for scanning the target object may include at least one, e.g., one, two, three, etc. In some embodiments, the non-zero diffusion factor for scanning the target object may include a first diffusion factor and a second diffusion factor. For example, the first diffusion factor may be 1000 and the second diffusion factor may be 1500.

In some embodiments, the processing device may determine at least one scanning sequence based on a plurality of target scanning directions, and scan the target object based on the at least one scanning sequence and the diffusion factors.

In 720, the target object may be scanned based on the at least two diffusion factors.

In some embodiments, the processing device may scan the target object based on the at least two diffusion factors. For example, if the diffusion factors includes a zero-valued diffusion factor and a non-zero diffusion factor of 1000, the scanning may be performed with diffusion factors of 0 and 1000, respectively. In some embodiments, the processing device may scan the target object based on the at least two non-zero diffusion factors.

In some embodiments, the processing device may obtain at least one set of scanning data by scanning the target object based on at least one scanning direction sequence and at least one of the first diffusion factor and the second diffusion factor.

In some embodiments, the processing device may scan the target object according to the at least one scanning direction sequence with a diffusion factor being the first diffusion factor, and designate the obtained scanning data as a first set of scanning data. A diffusion factor of the scanning data in the first set of scanning data may be the first diffusion factor.

In some embodiments, the processing device may scan the target object according to the at least one scanning direction sequence with the diffusion factor being the second diffusion factor, and designate the obtained scanning data as a second set of scanning data. A diffusion factor of the scanning data in the second set of scanning data may be the second diffusion factor.

In some embodiments, the processing device may scan the target object according to the at least one scanning direction sequence with the diffusion factors including the first diffusion factor and the second diffusion factor, and designate the obtained scanning data as a third set of scanning data. Diffusion factors of the scanning data in the third set of scanning data may include the first diffusion factor and the second diffusion factor.

In some embodiments, the target scanning directions in the scanning direction sequences corresponding to the first set of scanning data, the second set of scanning data, and the third set of scanning data may be partially or completely different. In some embodiments, the first set of scanning data, the second set of scanning data, and the third set of scanning data may be obtained based on a same scanning direction sequence.

In some embodiments, the target scanning direction in the scanning direction sequence corresponding to the first set of scanning data, the second set of scanning data, and the third set of scanning data may be completely different.

In some embodiments, images may be reconstructed based on the scanning data in the first set of scanning data and the second set of scanning data, and/or the third set of scanning data (i.e., a set of scanning data used for reconstructing the images includes the at least two different diffusion factors). The reconstructed images may be grouped according to different target scanning directions and different diffusion factors, and the fiber tracking may be performed separately based on each set of images to obtain different fiber tracking results, so as to finally determine a fiber tracking image of the target object. In some embodiments, the processing device may store the scanning data and/or the reconstructed images based on at least one of the scanning direction sequences and the diffusion factors, etc.

In some embodiments of the present disclosure, by generating different scanning sequences based on different non-zero diffusion factors, and scanning the target object based on the scanning sequences and the diffusion factors, more scanning data under different conditions may be obtained and categorized for storage, which increases data for combining in the subsequent determination of the fiber tracking image, thereby satisfying different observation requirements of the user and improving scanning flexibility.

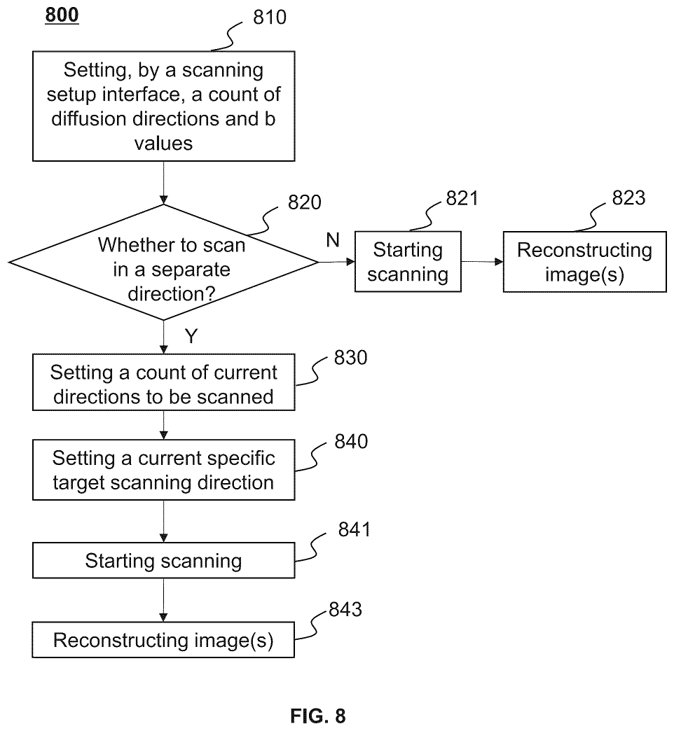

FIG. 8 is a schematic diagram illustrating a process 800 for diffusion tensor imaging according to some embodiments of the present disclosure.

As shown in FIG. 8, in some embodiments, the process 800 may include following operations.

In 810, a count of diffusion directions and b values may be set at a scanning setup interface. In some embodiments, the scanning setup interface may include an interface as shown in FIG. 9.

In some embodiments, the user may set the count of diffusion directions (i.e., a total count of diffusion directions) and the b values (i.e., diffusion factors) on the scanning setup interface. For example, as shown in FIG. 9, the total count of diffusion directions is set to 60, the count of b values (i.e., a number of b values) is set to 2, the two b values are 0 and 1000, and b value settings also include an average count setting. An average count of each b value is set to 1. In some embodiments, the diffusion gradient direction determination module 210 may determine diffusion gradient directions based on the total count of diffusion gradient directions. For example, if the total count of diffusion directions in FIG. 9 is 60, the determined diffusion gradient directions are 60.

In 820, whether to scan in a separate direction may be selected.

In some embodiments, the user may select whether to scan in the separate direction at the scanning setup interface. If the scan in the separate direction is selected, the scanning module 230 may proceed to operation 830. If the scan in the separate direction is not selected, the scanning module 230 may proceed to operation 821 to start scanning the target object (e.g., a human body, an animal, etc.) according to a default mode, i.e., scanning in all directions. Then, the image reconstruction module 240 may perform operation 823 to reconstruct image(s) based on scanning data obtained from the scanning. Operation 821 is similar to operation 841, and operation 823 is similar to operation 843. For example, as shown in FIG. 9, a separate direction scanning is selected.

In 830, a count of current directions to be scanned may be set.

In some embodiments, the user may set the count of current directions to be scanned at the scanning setup interface. For example, as shown in FIG. 9, the user may input 6 as the count of current directions to be scanned into an input box of the count of current scanning directions.

In 840, a current specific target scanning direction may be set.

In some embodiments, after the user sets the count of current directions to be scanned, the user may continue to set the current specific target scanning direction at the scanning setup interface. In some embodiments, the user may set the current specific target scanning direction by tapping on a gradient vector sphere graph, directly inputting the current specific target scanning direction into the input box, etc. For example, as shown in FIG. 9, the user may input numbers 7-12 of target scanning directions into the input box of the target scanning directions. That is, the diffusion gradient directions numbered 7-12 may be designated as the target scanning directions.

In some embodiments, after setting the current scanning directions, the scanning direction sequence determining module 220 may generate a scanning sequence based on the current target scanning directions, and the scanning module 230 may perform operation 841 to start scanning based on the generated scanning sequence of the current target scanning directions. Then, the image reconstruction module 240 may perform operation 843 to reconstruct image(s) based on the scanning data obtained from the scanning, and the image determination module 250 may obtain the fiber tracking image based on the reconstructed image(s). More descriptions regarding the generating the scanning sequence, scanning the target object, reconstructing the image(s), and obtaining the fiber tracking image may be found in FIG. 3 and relative descriptions, which are not repeated herein.

It should be noted that the descriptions of the processes 300, 400, 500, 600, and 700 are provided for the purposes of illustration, and are not intended to limit the scope of the present disclosure. For persons having ordinary skills in the art, various variations and modifications may be conducted under the teaching of the present disclosure. However, those variations and modifications may not depart from the protection of the present disclosure. For example, the process 500 may be included within the process 400, and the user instruction in operation 420 may include an instruction for the user to set the count of target scanning directions. As another example, the process 700 may be included within operation 310—operation 330.

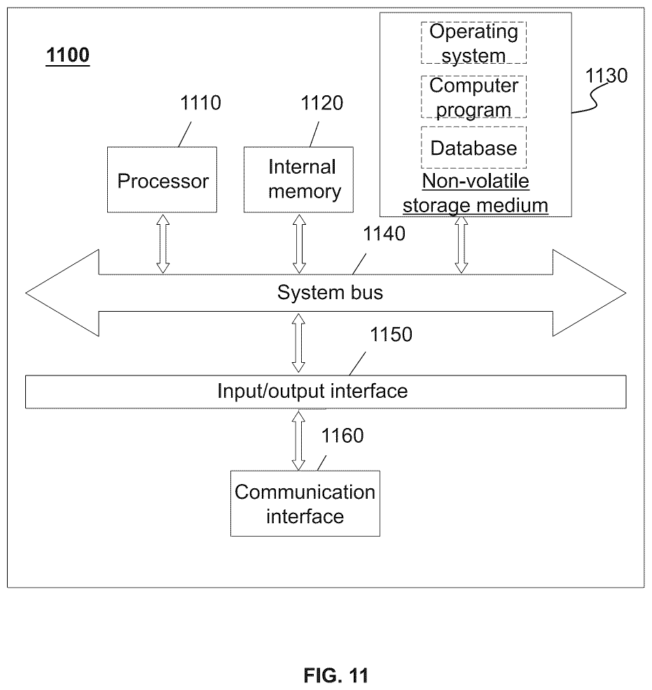

FIG. 11 is a schematic diagram illustrating a structure of a device 1100 for diffusion tensor imaging according to some embodiments of the present disclosure.

In some embodiments, as shown in FIG. 11, the device 1100 for diffusion tensor imaging may include a processor 1110, an internal memory 1120, a non-volatile storage medium 1130, a system bus 1140, an input/output interface 1150, and a communication interface 1160. In some embodiments, the device 1100 for diffusion tensor imaging may include a medical imaging device (e.g., the medical imaging device 110).

As shown in FIG. 11, the processor 1110, the memory, and the input/output interface 1150 may be connected via the system bus 1140, and the input/output interface 1150 may be connected to the communication interface 1160. The memory may include the internal memory 1120 and the non-volatile storage medium 1130.

The processor 1110 may be configured to provide computing and control capabilities. The non-volatile storage medium 1130 may store an operating system, a computer program, and a database. The database may be configured to store scanning data in different scanning directions. The internal memory 1120 may provide an operating environment for the operating system and the computer program in the non-volatile storage medium 1130. The input/output interface 1150 may be configured to exchange internal and external data. The communication interface 1160 may be configured to communicate with external terminals by a network connection. In some embodiments, the device 1100 for diffusion tensor imaging may include a display device and an input device. The display device may be configured to display an interactive interface with a user (e.g., a scanning setup interface, etc.), and may include various touch and/or non-touch display devices, such as a liquid crystal display (LCD), an electronic ink display, etc. The input device may be configured to receive user inputs, and may include various input devices, such as a touch layer covered by a display screen, a key, a trackball, a touchpad, a keyboard, a mouse, etc.

In some embodiments, the computer program stored in the non-volatile storage medium 1130 may be executed by the processor 1110 to implement the method for diffusion tensor imaging according to some embodiments of the present disclosure.

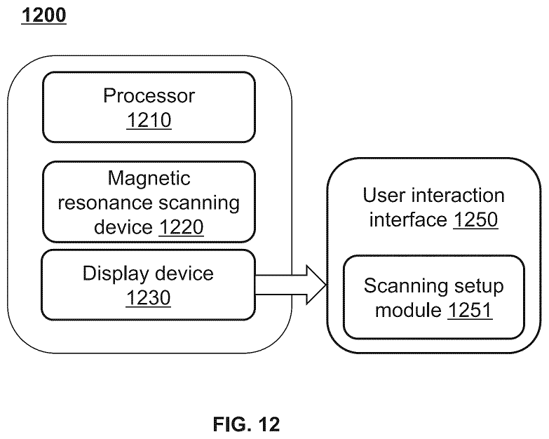

FIG. 12 is a schematic diagram illustrating a magnetic resonance imaging (MRI) system 1200 according to some embodiments of the present disclosure.

As shown in FIG. 12, in some embodiments, the MRI system 1200 may at least include a processor 1210, a magnetic resonance scanning device 1220, and a display device 1230.

In some embodiments, the processor 1210 may be configured to present a user interaction interface 1250 at the display device 1230.

In some embodiments, the processor 1210 may perform the method for diffusion tensor imaging according to some embodiments of the present disclosure based on operations (e.g., operations in any of the processes 300-600) of a user in the user interaction interface 1250.

In some embodiments, the magnetic resonance scanning device 1220 may be configured to scan a target object based on instructions of the processor 1210, thereby obtaining scanning data of the target object. In some embodiments, the magnetic resonance scanning device 1220 may include the medical imaging device 110, etc.

In some embodiments, the display device 1230 may be configured to display the user interaction interface 1250. The user interaction interface 1250 may at least include a scanning setup module 1251 configured to display a scanning setup interface, as shown in FIG. 9. In some embodiments, the user interaction interface 1250 may also include a display and editing interface for reconstructing images, etc.

In some embodiments, the scanning setup module 1251 may at least include a diffusion factor setup unit 910, a scanning mode setup unit 920, and a diffusion gradient direction setup unit 930 as shown in FIG. 9. The diffusion factor setup unit 910 may be configured to set at least one diffusion factor parameter, and the at least one diffusion factor parameter may at least include a count of diffusion factors and diffusion factor values. In FIG. 9, a count of b value (i.e., the count of diffusion factors) is 2, and the two b values (i.e., the diffusion factor values) are 0 and 1000, respectively. The scanning mode setup unit 920 may be configured to set scanning mode information, and the scanning mode information may at least include whether to scan in a separate direction, a current scanning direction, etc. The current scanning direction may include a count of current scanning directions, at least one target scanning direction, etc. In FIG. 9, a separate direction scanning has been selected to be performed, the count of current scanning directions is 6, and the at least one target scanning direction is 7-12, i.e., diffusion gradient directions numbered 7-12. The diffusion direction setup unit 930 may be configured to set the total count of diffusion directions. In FIG. 9, the total count of diffusion directions (i.e., the count of diffusion directions) is 60.

In some embodiments, the scanning setup module 1251 may further include a presentation unit. The presenting unit may be configured to display a distribution map of the diffusion gradient directions and the at least one target scanning direction selected by the user. For example, in a gradient vector sphere map shown in FIG. 10, directions shown by the vector arrows 1010-1060 are diffusion gradient directions, and a direction shown by the vector arrow 1020 is the target scanning direction selected by the user.

The possible beneficial effects of the embodiments of the present disclosure may include but not limited to the following: (1) by performing diffusion tensor scanning in separate directions and times, a plurality of diffusion gradient directions can be divided into a plurality of sequences for scanning according to needs, thereby reducing data volume obtained from each scanning, shortening a reconstruction time, decreasing image errors and artifacts, improving scanning stability, reducing post-processing requirements, and reducing reconstruction pressure. At the same time, the user may perform supplementary scanning in required directions according to a fiber tracking effect without re-scanning in all directions, which ensures the accuracy of the fiber tracking effect and improves the efficiency of image acquisition and reconstruction by diffusion tensor scanning. (2) The scanning can be performed based on required scanning direction(s) selected according to user requirements, which satisfies diversified requirements of different users, and avoids loss of time, experimental materials, and other materials caused by the re-scanning when the scanning fails over a long period of time, thereby saving costs and improving economic efficiency. (3) By obtaining correction parameters from scanning data of diffusion tensor imaging in at least one direction, and correcting a system state (e.g., eddy current, field drift, etc.) through the correction parameters at an interval between two scans, the accuracy of scanning results can be improved, and the problem of locating anomalous parameters only after finishing the scanning in all directions can be effectively solved, which improves the scanning efficiency. It should be noted that different embodiments may produce different beneficial effects, and in different embodiments, the beneficial effects that may be produced may be any one or a combination of any one or more of the above, or any other beneficial effect that may be obtained.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations, therefore, is not intended to limit the claimed processes and methods to any order except as may be specified in the claims. Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the invention. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A method for diffusion tensor imaging, implemented on a computing device having at least one processor and at least one storage device, the method comprising:
determining at least one diffusion gradient direction;
determining at least one scanning direction sequence based on the at least one diffusion gradient direction, wherein each of the at least one scanning direction sequence includes at least one target scanning direction; and
obtaining at least one set of scanning data by scanning a target object based on the at least one scanning direction sequence, wherein the at least one set of scanning data is used to determine a fiber tracking image of the target object.

2. The method of claim 1, wherein the determining at least one scanning direction sequence based on the at least one diffusion gradient direction includes:
obtaining a user instruction, wherein the user instruction includes indicative information of the at least one target scanning direction; and
determining the at least one scanning direction sequence based on the user instruction.

3. The method of claim 2, wherein the at least one diffusion gradient direction are determined based on a total count of the at least one diffusion gradient direction, and the method further comprises:
generating a distribution map of the at least one diffusion gradient direction in response to obtaining the total count of the at least one diffusion gradient direction; and
annotating, based on the user instruction, the at least one target scanning direction on the distribution map.

4. The method of claim 3, wherein the user instruction includes the at least one target scanning direction which is selected by a user on the distribution map.

5. The method of claim 1, wherein the determining at least one scanning direction sequence based on the at least one diffusion gradient direction includes:
obtaining a count of the at least one target scanning direction input by a user;
determining at least one set of candidate scanning directions from the at least one diffusion gradient direction based on the count of the at least one target scanning direction;
recommending the at least one set of candidate scanning directions to the user; and
determining the at least one scanning direction sequence in response to a selection of the user.

6. The method of claim 1, further comprising:
determining a current scanning direction;
obtaining current scanning data corresponding to the current scanning direction;
obtaining correction parameters based on the current scanning data; and
correcting, based on the correction parameters, initial parameters for scanning.

7. The method of claim 1, further comprising:
obtaining at least two diffusion factors, wherein the at least two diffusion factors include at least one zero-valued diffusion factor and at least one non-zero diffusion factor, or at least two non-zero diffusion factors; and wherein the scanning a target object further includes:
scanning the target object based on the at least two diffusion factors.

8. The method of claim 7, wherein the at least one non-zero diffusion factor includes a first diffusion factor and a second diffusion factor, and the scanning the target object based on the at least two diffusion factors includes:
obtaining the at least one set of scanning data by scanning the target object based on the at least one scanning direction sequence and at least one of the first diffusion factor and the second diffusion factor.

9. The method of claim 1, further comprising:
obtaining a plurality of reconstructed images by performing data reconstruction based on each of the at least one set of scanning data; and
determining the fiber tracking image of the target object based on reconstructed images of at least six directions among the plurality of reconstructed images.

10. The method of claim 9, wherein the reconstructed images of the at least six directions are obtained by reconstructing the at least one set of scanning data including a plurality of diffusion factor values in at least six different target scanning directions.

11. The method of claim 9, wherein the plurality of reconstructed images are stored based on the at least one scanning direction sequence and at least one diffusion factor.

12. The method of claim 9, wherein the determining the fiber tracking image of the target object based on reconstructed images of at least six directions among the plurality of reconstructed images includes:
obtaining at least one pre-selected reconstructed image selected by a user from the plurality of reconstructed images;
determining whether the at least one pre-selected reconstructed image satisfies a preset condition;
in response to determining that the at least one pre-selected reconstructed image satisfies the preset condition, determining the fiber tracking image of the target object based on the at least one pre-selected reconstructed image; or
in response to determining that the at least one pre-selected reconstructed image does not satisfy the preset condition, outputting a prompt message.

13. The method of claim 12, wherein the preset condition includes:
a count of diffusion gradient directions corresponding to the at least one pre-selected reconstructed image being greater than or equal to a preset value, and the diffusion gradient directions corresponding to the at least one pre-selected reconstructed image being uniformly distributed on a sphere surface.

14. (canceled)

15. A magnetic resonance imaging (MRI) system, comprising a processor and a magnetic resonance scanning device, wherein:
the processor is configured to present a user interaction interface at a display device; and
the magnetic resonance scanning device is configured to scan a target object based on an instruction from the processor, wherein
the user interaction interface includes a scanning setup module, and the scanning setup module at least includes a diffusion gradient direction setup unit, a diffusion factor setup unit, and a scanning mode setup unit, wherein
the diffusion gradient direction setup unit is configured to set a total count of at least one diffusion gradient direction;
the diffusion factor setup unit is configured to set diffusion factor parameters, the diffusion factor parameters at least including a count of diffusion factors and diffusion factor values; and
the scanning mode setup unit is configured to set scanning mode information, the scanning mode information at least including whether to scan in separate directions and a current scanning direction.

16. The MRI system of claim 15, wherein the scanning setup module further includes a presentation unit, and the presentation unit is configured to display a distribution map of at least one diffusion gradient direction and at least one target scanning direction selected by a user.

17-18. (canceled)

19. A non-transitory computer-readable storage medium storing computer instructions, wherein when a computer reads the computer instructions in the storage medium, the computer implements a method for diffusion tensor imaging, the method comprising:
determining at least one diffusion gradient direction;
determining at least one scanning direction sequence based on the at least one diffusion gradient direction, wherein each of the at least one scanning direction sequence includes at least one target scanning direction; and
obtaining at least one set of scanning data by scanning a target object based on the at least one scanning direction sequence, wherein the at least one set of scanning data is used to determine a fiber tracking image of the target object.

20. The MRI system of claim 15, wherein the processor is further configured to:
determine at least one diffusion gradient direction;
determine at least one scanning direction sequence based on the at least one diffusion gradient direction, wherein each of the at least one scanning direction sequence includes at least one target scanning direction; and
obtain at least one set of scanning data by scanning the target object based on the at least one scanning direction sequence, wherein the at least one set of scanning data is used to determine a fiber tracking image of the target object.

21. The MRI system of claim 20, wherein to determine the at least one scanning direction sequence based on the at least one diffusion gradient direction, the processor is further configured to:
obtain a user instruction, wherein the user instruction includes indicative information of the at least one target scanning direction; and
determine the at least one scanning direction sequence based on the user instruction.

22. The MRI system of claim 20, wherein to determine the at least one scanning direction sequence based on the at least one diffusion gradient direction, the processor is further configured to:
obtain a count of the at least one target scanning direction input by a user;
determine at least one set of candidate scanning directions based on the at least one diffusion gradient direction based on the count of the at least one target scanning direction;
recommend the at least one set of candidate scanning directions to the user; and
determine the at least one scanning direction sequence in response to a selection of the user.

23. The MRI system of claim 20, wherein the processor is further configured to:
determine a current scanning direction;
obtain current scanning data corresponding to the current scanning direction;
obtain a correction parameter based on the current scanning data; and
correct, based on the correction parameter, an initial parameter of a current scanning.
